(19) United States
(12) Patent Application Publication
(10) Pub. No.: US 2005/0220949 A1
(43) Pub. Date: Oct. 6, 2005

(54) Highly bioefficient isoflavone-phospholipid molecular complexes and methods of making and using the same

(76) Inventor: Blas Arzubi, Rosario (AR)

Correspondence Address: COLLEN IP, THE HOLYOKE MANHATTAN BUILDING, 80 SOUTH HIGHLAND AVENUE, OSSINING, NY, 10562, US

(21) Appl. No.: 10/813,969

(22) Filed: Mar. 31, 2004

(51) Int. Cl. C12N 5/00

(52) U.S. Cl. 426/480; 426/601; 426/481

(57) ABSTRACT

The present application describes highly bioefficient isoflavone-phospholipid molecular complexes, which are prepared by co-processing raw phospholipid materials with isoflavone-rich plant materials.

### FIELD OF THE INVENTION

This invention relates generally to highly bioefficient isoflavone-phospholipid molecular complexes, methods of obtaining the same through co-processing of raw phospholipid materials with isoflavone-rich plant materials, and methods of using the same.

### BACKGROUND OF THE INVENTION

U.S. Pat. No. 5,453,523 and U.S. Pat. No. 5,703,255 indicate that the term “lecithin,” as used in the fats and oils industry, refers to a mixture of phosphatides that may include phosphatidylcholine (PC), phosphatidylethyl alcoholamine (PE). phosphatidylinositol (PI), phosphatidylserine (PS). phosphatidic acid (PA). and others. Lecithin has been used in the scientific literature to refer mostly to PC. The term “cephalin” has been used to refer to PE, mixtures of PE, and the non-choline phosphatides.

Processes for the separation of lecithin from contaminating substances are described, for example, in the article by Szuhaj, B. F. (ed.) Lecithins, American Oil Chemist's Society, Champaign, Ill. 1989. Recognition of the unique properties and possible uses of the individual phosphatide components of lecithin, particularly PC, and the adverse effects in certain applications of contaminating non-choline phosphatides in PC enriched fractions, has stimulated the search for improved methods of PC purification from lecithin.

Isoflavones are plant chemicals which occur largely in members of the Leguminosae plant family. They are based on a simple diphenolic ring structure as described for example by Carlson et al (1980) Journal of Chromotography, 198, 193-197. Over 700 different isoflavones have been described, and these display a range of biological functions both within the plant and within animals and humans which eat the isoflavone-containing plants.

A small sub-group of isoflavones (daidzein, genistein, biochanin A, formononetin, and glycitein) are distinguished by their ability to bind to estrogen receptors on both animal and human cells. This is due to the close similarity of the steric structure of the diphenolic rings of isoflavones with the steroidal ring structure of estrogens such as estradiol, estrone, and estriol. Although having substantially lower binding affinity to the receptor compared to steroidal estrogens, estrogenic isoflavones are weakly estrogenic. This group also exhibits a range of biological functions in both animal and human cells, which appear to be independent of the estrogen receptor, including anti-oxidant, diuretic, anti-spasmolytic, and anti-cancer effects. These interesting functions with their potential therapeutic benefits have recently brought this particular group of isoflavones to the attention of medical researchers.

The glycosidic form of isoflavones (either alone or in their malonyl or acetyl forms) is water-soluble and is the predominant form for plant isoflavones to facilitate transport and storage. The glycosidic form also provides enhanced stability to degradative factors such as heat, oxidation, and ultraviolet irradiation. The aglycone form, although more biologically active, it is substantially water-insoluble.

U.S. Pat. No. 5,670,632, granted to Chaihorsky, describes a process for recovering isoflavones from a soy extract obtained through the previous extraction of fat and protein with a hydrocarbon such as n-hexane; then dissolving in an aqueous solvent, and finally desorbing the 7-glycosyl-isoflavones with a ground highly polar cationic exchange resin and an appropriate eluent such as acidic 86% ethyl alcohol.

U.S. Pat. No. 5,679,806, granted to Zheng, et al., refers to the isolation and purification of isoflavones starting from soy molasses, forming an alcoholic extract, and adsorbing it through a column using a reverse phase matrix in combination with a step gradient elution over the column. The resulting fraction may be hydrolized and/or dried to reach crystallization.

U.S. Pat. No. 6,033,714, granted to Gugger, et al., deals with producing isoflavone fractions by a treatment of an aqueous alcohol extract of defatted soybean flakes, starting preferably with soy molasses alternatively with soy whey, which is then subjected to ultrafiltration in order to produce a permeate which then passes through a column containing an adsorbing resin.

U.S. Pat. No. 4,414,157, granted to Iwama, et al., describes a process for the purification of crude glyceride oil compositions containing a glyceride oil and phospholipid with an organic solvent, bringing the diluted crude glyceride oil composition under pressure and into contact with a capillary semipermeable membrane to concentrate the miscella to a predetermined level, and then bringing the preliminarily concentrated miscella into contact with a tubular semipermeable membrane to concentrate the miscella to a higher level.

The above-noted examples show that lecithin (phospholipids) and isoflavones are obtained and purified separately. The co-processing of raw phospholipid materials with isoflavone-rich plant materials has not been described. Furthermore, there has been no suggestion that such novel co-processing would provide the novel, highly bioefficient isoflavone-phospholipid molecular complexes discovered by the present inventors.

### SUMMARY OF THE INVENTION

The present invention provides novel isoflavone-phospholipid molecular complexes.

The present invention also provides a novel method of obtaining isoflavone-phospholipid molecular complexes.

The present invention also provides a novel food ingredient, dietary supplement, cosmetic composition, or pharmaceutical composition containing an isoflavone-phospholipid molecular complex.

The present invention also provides a novel method of treating using an isoflavone-phospholipid molecular complex.

### BRIEF DESCRIPTION OF THE DRAWINGS

FIG. 1: Diagram of the process for obtaining Isoflavone-Phospholipid Molecular Complexes.

FIG. 2: Reverse-phase HPLC chart obtained of Isoflavone-Phospholipid Molecular Complex “A”.

FIG. 3: Reverse-phase HPLC chart obtained of Isoflavone-Phospholipid Molecular Complex “B”.

FIG. 4: Reverse-phase HPLC chart obtained of Isoflavone-Phospholipid Molecular Complex “C”.

### DETAILED DESCRIPTION OF THE INVENTION

In an embodiment, the present invention provides novel isoflavone-phospholipid molecular complexes. These complexes are formed by co-processing raw phospholipid materials with isoflavone-rich plant materials. The raw phospholipid materials are obtained from vegetable sources, animal sources, or a combination thereof. The isoflavone-rich plant materials are extracted from any kind of isoflavone-containing plant matter, preferably one or more vegetable sources.

Preferably, the raw phospholipid material is selected from: soy gums, soy lecithins, deoiled soy lecithin, sunflower lecithin, deoiled sunflower lecithin, egg lecithin, deoiled egg lecithin, egg yolk, brain lecithin, and deoiled brain lecithin. Even more preferably, the raw phospholipid material is selected from the group: soy gums and soy lecithins.

The starting isoflavone-rich plant materials are preferably selected from: Indian liquorice (Abrus precatorius); various species of Acacia (e.g., A. aneura, A. cibaria, A. longifolia, and A. oswaldii); ground nut (Apio tuberosa); ground pea (Arachis hypogea); milk vetch (Astragalus edulis); marama bean (Bauhinia esculenta); sword bean (Cajanus cajan indicus); jack bean (Canavalia ensiformis); sword bean (Canavalia gladiata); seaside sword bean (Canavalia rosea); various species of Cassia (e.g., C. floribunda, C. laevigata, and C. occidentalis); carobbean (Ceratonia siliqua); chick pea (Cicer arietinum); yebnut (Cordeauxia edulis); various species of Crotalaria (e.g., C. labumifolia, and C. pallida); cluster bean (Cyamopsis psoralioides); tallow tree (Detariaum senegalense); sword bean (Entada scandens); balu (Erythrina edulis); soyabean (Glycine max); wild soya (Glycine soja); inga (Ingaedulis); Polynesian chestnut (Inocarpus fagifer); hyacinth bean (Lablab purpureus); grass pea or Indian vetch (Lathyrus sativus); cyprus vetch (Lathyrus ochrus); lentil (Lens culinaris); jumping bean (Leucaenal eucocephala); various species of Lupinus (e.g., L. albus, L. luteus, L. angustifolium, L. mutabilis, and L. cosentinii); ground bean (Macotylma geocarpa); horse gram (Macrotyloma uniflorum); alfalfa (Medicago sativa); velvet bean (Mucuna pruriens); yam beans (Pachyrhyzuz erosus, P. tuberosus); African locust bean (Parkia clappertoniana); Parkia speciosa; oil bean tree (Pentaclethra macrophylla); various species of Phaseolus (e.g., P. acutifolius, P. vulgaris, P. luntus, P. coccineus, P. adenathus, P. angulris, P. aureus, P. calcaratus, P. mungo, and P. polystachyus); garden pea (Pisum sativum); djenko bean (Pithecolobium lobatum); mesquite (various Prosopis spp.); goa bean (Psophocarpus scandens, P. tetragonolobus); various species of Psoralea; Sesbania bispinosa; yam bean (Sphenostylis stenocarpa); tamarind (Tamarindus indica); Trifolium pretense; Trifolium subterranean; fenugreek (Trigonella foenum-graecum); vetches; various species of Vivia (e.g, V. sativa, V. atropurpurea, V. ervilia, and V. monantha); broad bean (Vicia faba); black grani (Vigna mungo); various species of Vigna (e.g., V. radiata, V. aconitifolia, V. adanatha, V. angularus, V. tribolata, V. umbelata, and V. unguiculata); and, earth pea (Voandzeia subterranea).

Preferably, the starting isoflavone-rich plant material is at least one material selected from: soybeans, soy flours (i.e., fill-fat soy flours, partially-defatted soy flours, and defatted soy flours), soy isoflavones concentrates, red clover isoflavones concentrates, and soy grits. More preferably, the starting isoflavone-rich plant material is soy flour.

The phospholipid concentration in the raw phospholipid materials can be anywhere from detectable limits to 0.1, 1, 5, 10, 15, 20, 25, 30, 35, 40, 45, 50, 55, 60, 65, 70, 75, 80, 85, 90, 95, to 100%, by weight. Preferably, the phospholipid concentration in the raw phospholipid materials is from about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95 to 98%, by weight. The isoflavone concentration in the isoflavone-rich plant material can be anywhere from detectable limits to 0.1, 1, 5, 10, 15, 20, 25, 30, 35, 40, 45, 50, 55, 60, 65, 70, 75, 80, 85, 90, 95, to 100%, by weight. Preferably, the isoflavone concentration in the isoflavone-rich plant material is from about 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, to 80%, by weight.

In another embodiment, the present invention provides a method of preparing isoflavone-phospholipid molecular complexes, comprising: mixing raw phospholipid materials and isoflavone-rich plant materials, contacting the mixture with an alcohol to extract the complexes, and filtering. Preferably, the filtrate is then dried.

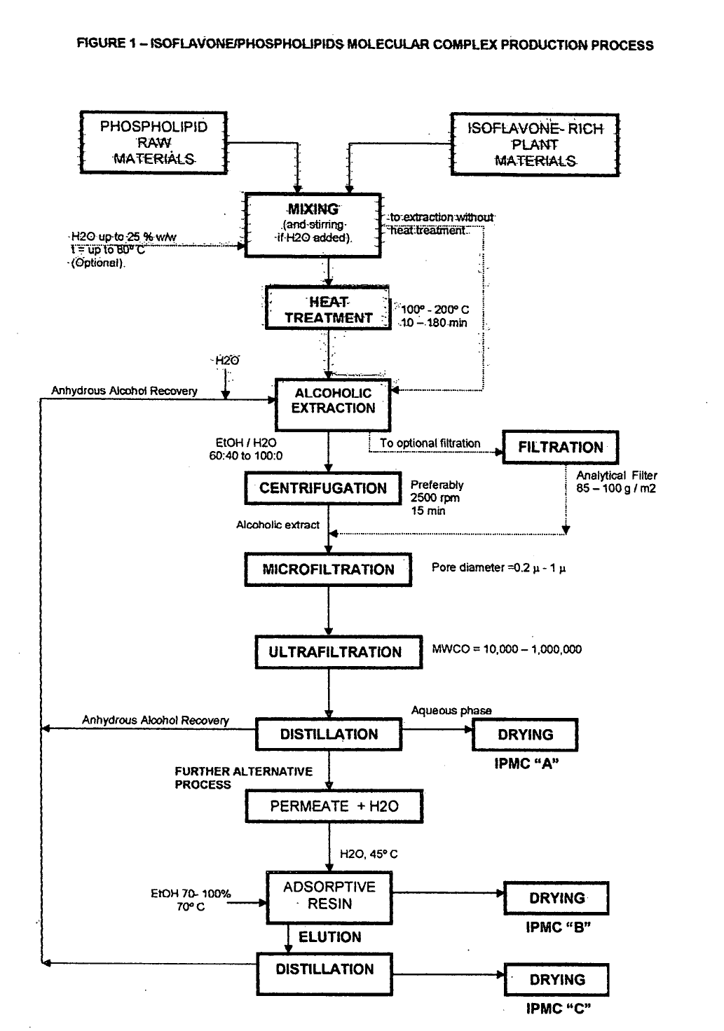

The flow diagram in FIG. 1 illustrates a number of preferred procedures for obtaining said isoflavone-phospholipid molecular complexes. First, raw phospholipid materials are mixed with isoflavone-rich plant materials. This can generally be accomplished by contacting the starting materials in mixer, though one of ordinary skill in the art would recognize that there are numerous ways to mix the starting materials.

After mixing, the resulting mixture may optionally be hydrated. The amount of water is preferably from about 1, 2, 3, 4, 5, 6, 7, 8, 9, 10, 11, 12, 13, 14, 15, 16, 17, 18, 19, 20, 21, 22, 23, 24, to 25% w/w. Hydrating can be performed at room temperature or up to a temperature not over about 80° C. (e.g., about 35, 40, 45, 50, 55, 60, 65, 70, 75, or 80° C.).

After mixing and optionally hydrating, the mixture may optionally be heat-treated, preferably at a temperature from about 100, 110, 120, 130, 140, 150, 160, 170, 180, 190, to 200° C. for about 10, 20, 30, 40, 50, 60, 70, 80, 90, 100, 110, 120, 130, 140, 150, 160, 170, to 180 minutes. In a preferred embodiment, the mixture is heat-treated at 170° C.

If the mixture is heat-treated, then it is preferably cooled down to about 25, 30, 35, 40, 45, 50, 55, to 60° C. and then contacted (e.g., stirred) with an aqueous alcohol. The heat-treated mixture is preferably cooled to about 40, 45, 50, 55, to 60° C. Preferably, the alcohol is selected from: methyl alcohol, ethyl alcohol, and isopropyl alcohol, preferably aqueous alcohol, more preferably ethyl alcohol. Preferably, the heat-treated mixture is contacted with aqueous ethyl alcohol in an approximate relation between 60:40, 65:35, 70:30, 75:25, 80:20, 85:15, 90:10, to 100:0 alcohol to water v/v, at a temperature of about 25, 30, 35, 40, 45, 50, 55, to 60° C.

If the mixture is not heat-treated, then it is contacted (e.g., stirred) directly with an alcohol, preferably aqueous alcohol. Preferably, the alcohol is selected from: methyl alcohol, ethyl alcohol, and isopropyl alcohol, more preferably ethyl alcohol. Preferably, the non-heat-treated mixture is contacted with aqueous ethyl alcohol in an approximate relation between 60:40, 65:35, 70:30, 75:25, 80:20, 85:15, 90:10, to 100:0 alcohol to water v/v, at a temperature of about 25, 30, 35, 40, 45, 50, 55, to 60° C.

After contacting, the mixture then needs to be filtered in order to isolate the new isoflavone-phospholipid molecular complexes. It is preferred to use a series of filtrations in order to isolate complexes.

A first filtration is preferably via centrifugation. Preferably, the alcoholic mixture is centrifuged at about 1500, 2000, 2500, 3000, 3500 rpm for about 1, 2, 3, 4, 5, 10, 15, 20, 25, or 30 minutes to separate the solids and form an alcoholic extract. Preferably, the alcoholic mixture is centrifuged at about 2500 rpm for about 15 minutes.

Alternatively, instead of centrifugation, the alcoholic mixture may be filtrated through a filtration medium to form an alcoholic extract. A filter such as those supplied by Schleicher & Schuell, Whatmam, Munktell and Macherey-Nagel in a range between 85, 90, 95, to 100 g/m² can be used.

A second filtration is preferably a microfiltration. This filtration is preferably conducted using a membrane with a pore diameter in a range of about 0.2, 0.3, 0.4, 0.5, 0.6, 0.7, 0.8, 0.9, to 1μ to separate some of the remaining solids. This filtration results in an alcoholic permeate.

A third filtration is preferably an ultrafiltration (UF). It is preferred to filter the alcoholic permeate through a membrane with a molecular weight cut off (MWCO) of 10,000, 20,000, 30,000, 40,000, 50,000, 100,000, 150,000, 200,000, 250,000, 300,000, 400,000, 500,000, 600,000, 700,000, 800,000, 900,000, to 1,000,000. Most soluble proteins are retained by the membrane, leaving an ultrafiltrated alcoholic permeate. Preferably, the MWCO of the membrane used for ultrafiltration is 150,000.

After the alcoholic mixture has been sufficiently filtered, it is preferable to remove the alcohol portion. Distillation allows for recovery of the alcohol for recycling back to the contacting process.

It is also preferable to dry the distillate in order to recover the novel isoflavone-phospholipid molecular complexes. The drying can be performed by techniques known to those of skill in the art. Preferred methods of drying include, but are not limited to vacuum drying, spray drying, and fluid bed.

An alternative process for concentrating and separating the new isoflavone-phospholipid molecular complexes, comprises: adding water to the residue after distilling off the alcohol and then passing this mixture through an adsorptive resin. There are a number of resins which may be used in this stage including, but not limited to, a divinylbenzene copolymer, ethylvinylbenzene-divinylbenzene copolymer, non-ionic styrene-divinylbenzene copolymer; ionic polystyrene; non-ionic polystyrene; nonionic ethylvinylbenzene-divinylbenzene copolymer; and ionic styrene-divinylbenzene copolymer.

Different isoflavone-phospholipid molecular complexes can be obtained from the resin via a warm water wash or a warm alcohol wash. After the above aqueous fraction has passed through the resin, a different complex can be obtained by washing the resin with water at about 45° C. This aqueous portion is then dried according to techniques known by those skilled in the art to obtain a different isoflavone-phospholipid molecular complex. The resin can then be eluted at about 30, 35, 40, 45, 50, 55, 60, 65 to 70° C. (preferably 70° C.) with 70, 75, 80, 85, 90, 95, to 100% alcohol by volume, preferably ethyl alcohol. The alcohol can then be removed (e.g., distilled off) and another different isoflavone-phospholipid molecular complex obtained.

A preferred resin is “Amberlite” XAD-16 polymeric adsorbent sold by Rohm and Haas Company at the Independence Mall West, Philadelphia, Pa. 19105. The manufacturer describes this resin as a non-ionic hydrophobic, crosslinked polymer which derives its adsorptive properties from its macroreticular structure containing both a continuous polymer phase and a continuous pore phase. The physical properties of “Amberlite” XAD-16 are described by this manufacturer as follows:

| | |
|---|---|
| Matrix | Macroreticular aliphatic crosslinked polymer |
| Physical form | White transluscent beads |
| Specific gravity | 1.015 to 1.025 g/ml |
| Particle size | 0.56-0.71 mm |
| Surface Area | ≧800 m²/g minimum |
| Porosity | ≧55% (vol/vol) minimum |

In another embodiment, the present invention provides a novel food ingredient, dietary supplement, cosmetic, or pharmaceutical composition, comprising: an isoflavone-phospholipid molecular complex of the present invention.

In another embodiment, the present invention provides a novel method, comprising: administering a therapeutically effective amount of an isoflavone-phospholipid molecular complex of the present invention to a person in need or desiring thereof.

Preferably, the isoflavone-phospholipid molecular complexes of the present invention are prepared in a dosage form selected from: a concentrate, dry powder, liquid, capsule, pellet, tablet, coated tablet, pill, a food ingredient, food supplement, health bar, soft gel, intranasal spray, intranasal drops, suspensions, syrups, sterile injectable solutions, parenteral solution, intravenous solution, milk, ointment, white or colored cream, serum, stick, paste, lotion, or similar dosage form.

Preferably, from about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900 to 3000 mg of at least one of the present isoflavone-phospholipid molecular complexes is administered daily to a patient in need or desiring thereof. More preferably, from about 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 to 500 mg of at least one of the present isoflavone-phospholipid molecular complexes is administered daily to a patient in need or desiring thereof.

Preferably at least one of said isoflavone-phospholipid molecular complexes is administered in a therapeutically effective amount to treating a disease selected from: premenstrual syndrome, including fluid retention, cyclical mastalgia, and dysmenorrhoea; menopausal syndrome including hot flushes, anxiety, depression, headaches, mood swings, night sweats, and urinary incontinence; Buergers Disease; Reynaud's Syndrome; Reynaud's Phenomenon; angina pectoris; coronary artery spasm; migrane headaches; hypertension; benign prostatic hypertophy; breast cancer; endometrial cancer; large bowel cancer; ovarian cancer; prostatic cancer; testicular cancer; uterine cancer; atherosclerosis; Alzheimers disease; dementia; cognitive dysfunction; inflammatory diseases including inflammatory bowel disease, Crohns disease, ulcerative colitis; alcoholemia; cirrhosis; delirium tremens; osteoporosis; rheumatic diseases including rheumatoid arthritis; acne; baldness including male pattern baldness (alopecia hereditaria); psoriasis and diseases associated with oxidant stress including cancer, myocardial infarction stroke, arthritis, sunlight induced skin damage, and cataracts. Treatment or treating, as used herein, covers the treatment of a disease-state in a mammal, particularly in a human, and includes prophylaxis, amelioration, defense against, prevention, reducing the predisposition towards, or a combination thereof.

In another embodiment, the present invention provides a novel method of treating or reducing the predisposition to symptoms associated with a disease, comprising: administering a therapeutically effective amount of at least one of isoflavone-phospholipid molecular complex, wherein the disease is selected from: menopause, cancer, Alzheimer's disease, atherosclerosis, hypercholesterolemia, dementia, cognitive dysfunction, osteoporosis, pre-menstrual syndrome, prostate diseases, and alcoholemia.

The cosmetic compositions of the present invention that comprise at least one isoflavone-phospholipid molecular complex are preferably in a form selected from: aqueous solution, aqueous-alcoholic solution, oily solution, oil-in-water, water-in-oil, multiple emulsion, an aqueous gel, oily gel, liquid product, pasty product, solid anhydrous product, a dispersion of oil in an aqueous phase using polymer nanoparticles such as nanospheres and nanocapsules, ionic lipid vesicles, non-ionic lipid vesicles, and a combination of ionic and non-ionic lipid vesicles. Preferably, the cosmetic compositions further comprise: contain common adjuvants selected from: hydrophilic or lipophilic gelling agents, hydrophilic or lipophilic active agents, preserving agents, antioxidants, solvents, fragrances, fillers, screening agents, pigments, chelating agents, odor absorbers, and dyestuffs, preferably in a concentration from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, to 20% w/w.

The following examples should not be interpreted as limitative but as merely illustrative of the present invention.

### EXAMPLES

### Example 1

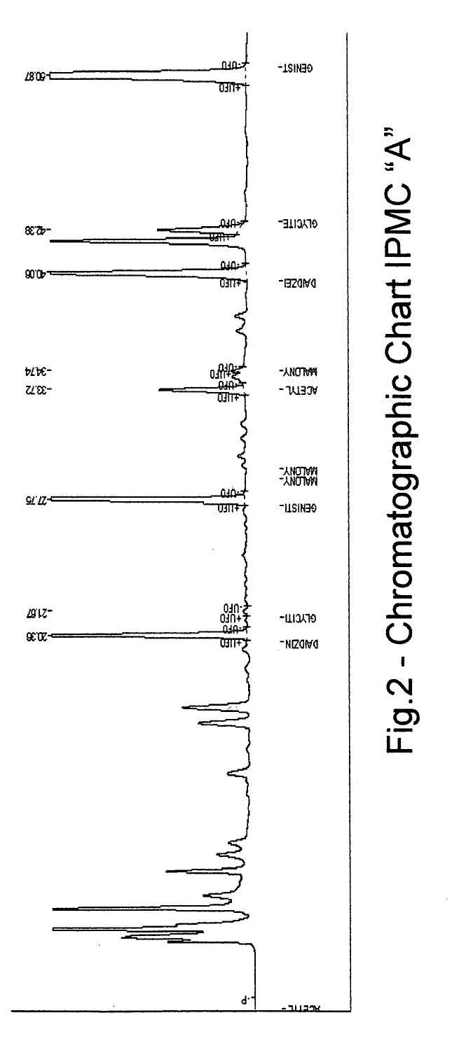

Soy gums (100 g) were added to defatted soy flour (1 kg) in a mixer. The mixture was heat-treated at a temperature of 170° C. for about an hour. The heat-treated mix was cooled down to 60° C. and then stirred with 90:10 aqueous ethyl alcohol. The mixture was centrifuged for 15 minutes at 2500 rpm to separate the solids and obtain an aqueous alcoholic extract. The resulting aqueous alcoholic extract was microfiltrated using a membrane with a pore diameter of 0.45μ. The microfiltrate was ultrafiltrated using a membrane with a MWCO of 150,000. The ethyl alcohol was distilled off from the ultrafiltrate. The resulting aqueous fraction was then dried to get a dark brown product. The resulting isoflavone-phospholipid molecular complex was called IPMC “A”. FIG. 2 is the reverse-phase HPLC chart of IPMC “A”. Although several components were identified by comparison with isoflavones standards, others were undetermined.

A series of animal studies was made to measure bioefficacy, on the basis of the effects on oxidative stress in CNS, of IPMC “A” in comparison with commercial isoflavone concentrates.

Eight-week-old Wistar rats (n=24) were randomized into four groups and treated for 30 days. All the groups were fed with an isoflavone-free diet, especially prepared for these studies (48% cheese whey protein concentrate; 39.8% whole-bran wheat flour; and, 12.2% whole milk).

Group I, “control group”, was fed only with isoflavone-free diet.

Groups II, III, and IV were i.p. injected with 2.5 mg sodium metavanadate (MVNa)/kg/day, in order to induce oxidative stress. These injections were carried out in the last 5 days of the treatment.

Group III was supplemented with 250 mg isoflavones aglycones-equivalent/kg diet through commercial isoflavone concentrates.

Group IV was supplemented with 250 mg isoflavones aglycones-equivalent/kg diet through the new isoflavone-phospholipid molecular complex IPMC

After the injection of MVNa, the rats were assigned to behavior studies measuring the spontaneous activity in an open field as well as motion coordination and strain resistance, this last parameter through a rota-rod instrument.

In Group II, a decrease was observed in stay time of rats in the motion rota-rod instrument. In the same way, rats from Group II showed depressed motion activity when measuring their average motion activity in an open field.

When comparing Group II with Group III, there was no noticeable change. In Group IV, an increased stay time in the rota-rod instrument test was observed, and the rats improved their motion activity during open field tests. The parameters measured in rats from Group IV were notoriously closer to those obtained in the Control Group I.

The results obtained suggest that commercial isoflavones at a dose of 250 mg do not control oxidant stress. Surprisingly, the above results show that the present isoflavone-phospholipid molecular complexes (i.e., IPMC “A”) controls the oxidative stress induced in the CNS of rats by sodium metavanadate (MVNa). Thus, it was concluded that novel newly isoflavone-phospholipid molecular complex, IPMC “A,” is outstandingly bioefficient.

### Example 2

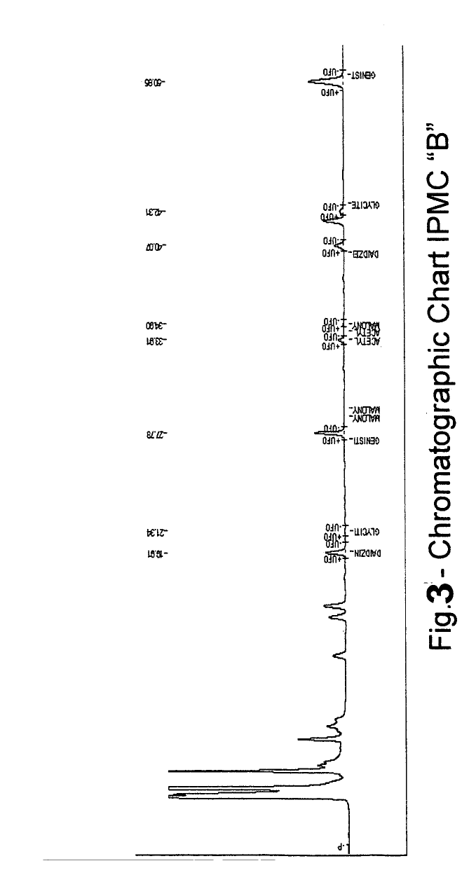

Egg yolk (25 g) was added to full-fat soy flour (1 kg) in a mixer. The mixture was heat-treated at a temperature of 170° C. for about an hour. The heat-treated mixture was cooled down to 60° C. and then stirred with 80:20 aqueous ethyl alcohol. The resulting mixture was centrifuged for 15 minutes at 2500 rpm to separate the solids and get an aqueous alcoholic extract. The extract was microfiltrated using a membrane a pore diameter of 0.45μ. The microfiltrate was then ultrafiltrated using a membrane with a MWCO of 150,000. The ethyl alcohol was distilled off from the ultrafiltrate. To the distillate was added water (500 ml). This aqueous fraction was passed through an adsorptive resin “Amberlite” XAD-16. After the aqueous fraction had passed through the resin, the resin was washed with water at 45° C. The permeate obtained was then dried. Isoflavone-phospholipid molecular complex, IPMC “B,” was obtained. FIG. 3 is the corresponding HPLC chart.

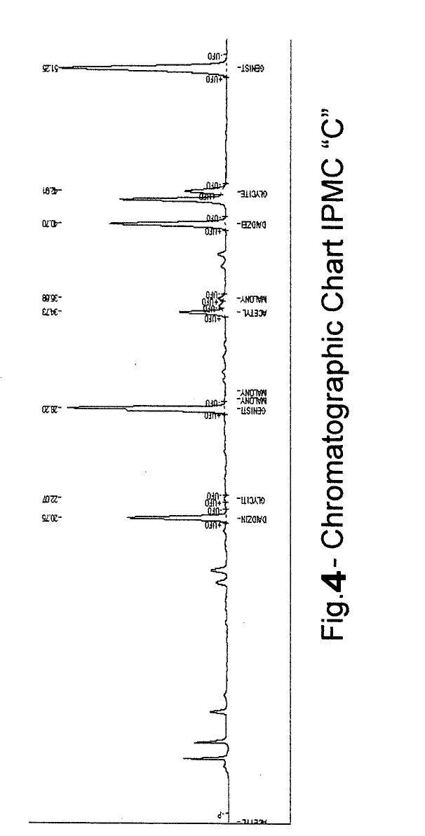

The retentate in the resin was eluted at 70° C. with 80%-100% ethyl alcohol. The alcoholic fraction was distilled and dried, and isoflavone-phospholipid molecular complex, IPMC “C,” was obtained. FIG. 4 is the corresponding HPLC chart.

A series of animal studies was made to measure the bioefficacy of new isoflavone-phospholipid molecular complexes IPMC “B” and IPMC “C” through the quantification of their capability as anti-oxidant agents in the CNS of rats.

Eight-week-old Wistar rats (n=30) were randomized into five groups and treated for 30 days. All the groups were fed with an isoflavone-free diet, especially prepared for these studies (48% cheese whey protein concentrate; 39.8% whole-bran wheat flour; and, 12.2% whole milk).

Group I “control group,” was fed only with isoflavone-free diet.

Groups II, III, IV and V were i.p. injected with 2.5 mg sodium metavanadate (MVNa)/kg/day, in order to induce oxidative stress. These injections were carried out in the last 5 days of the treatment.

Group III was supplemented with 250 mg isoflavones aglycones-equivalent/kg diet through commercial isoflavone concentrates.

Group IV was supplemented with 250 mg isoflavones aglycones-equivalent/kg diet through the new isoflavone-phospholipid molecular complex IPMC

Group V was supplemented with 250 mg isoflavones aglycones-equivalent/kg diet through the new isoflavone-phospholipid molecular complex IPMC “C”.

After the injection of MVNa, the rats were assigned to behavior studies measuring the spontaneous activity in an open field as well as motion coordination and strain resistance, this last parameter through a rota-rod instrument.

When comparing Group II with Group III, there was no noticeable change. In Groups IV and V, however, an increased stay time in the rota-rod instrument test was observed, and the rats improved their motion activity during open field tests. The parameters measured in rats from Groups IV and V were notoriously closer to those obtained in the Control Group I.

The results obtained suggest that commercial isoflavones at a dose of 250 mg do not control oxidant stress. Surprisingly, the above results show that new isoflavone-phospholipid molecular complexes IPMC “B” and IPMC “C” are comparable with those of the Control Group I, which evidenced no neuronal injury. Thus, IPMC “B” and IPMC “C” control the oxidative stress induced in the CNS of rats by sodium metavanadate (MVNa) and are outstandingly bioefficient.

Numerous modifications and variations of the present invention are possible in light of the above teachings. It is therefore to be understood that within the scope of the appended claims, the invention may be practiced otherwise than as specifically described herein.

1. A novel isoflavone-phospholipid molecular complex.

2. A complex according to claim 1, wherein the complex is formed by the method, comprising: co-processing raw phospholipid materials with isoflavone-rich plant materials, wherein the raw phospholipid materials are obtained from vegetable sources, animal sources, or a combination thereof and the isoflavone-rich plant materials are extracted from a vegetable source.

3. A complex according to claim 2, wherein the raw phospholipid material is selected from: soy gum, soy lecithin, deoiled soy lecithin, sunflower lecithin, deoiled sunflower lecithin, egg lecithin, deoiled egg lecithin, egg yolk, brain lecithin, and deoiled brain lecithin and the starting isoflavone-rich plant material is at least one material selected from: soybean, full-fat soy flour, partially-defatted soy flour, and defatted soy flour, soy isoflavones concentrate, red clover isoflavones concentrate, and soy grits.

4. A complex according to claim 3, wherein the raw phospholipid material is selected from the group: soy gum and soy lecithin and the starting isoflavone-rich plant material is soy flour.

5. A complex according to claim 2, wherein the phospholipid concentration in the raw phospholipid materials is from about 0.1-98%, by weight and the isoflavone concentration in the starting isoflavone-rich plant material is from about 0.1-80%, by weight.

6. A method of preparing isoflavone-phospholipid molecular complexes, comprising: (a) mixing raw phospholipid materials and isoflavone-rich plant materials, (b) contacting the mixture with an alcohol to extract the complexes, and (c) filtering.

7. A method according to claim 6, further comprising: (d) drying the filtrate.

8. A method according to claim 6, further comprising: (a₁) hydrating the resulting mixture from (a) with about 1-25% water w/w prior to performing (b).

9. A method according to claim 6, further comprising (a₂) heat-treating the mixture from (a) at a temperature of about 100-200° C. for about 10-180 minutes, prior to performing (b).

10. A method according to claim 9, wherein the heat-treating is performed at about 170° C.

11. A method according to claim 9, wherein heat-treated mixture is cooled to about 40-60° C. prior to performing (b).

12. A method according to claim 6, wherein (b) is performed by contacting the resulting mixture with an alcohol selected from: methyl alcohol, ethyl alcohol, and isopropyl alcohol.

13. A method according to claim 12, wherein the alcohol is aqueous ethyl alcohol in an alcohol to water range of about 60:40 to 100:0, v/v.

14. A method according to claim 6, wherein (c) is performed via an ultrafiltration with a membrane having a MWCO of about 10,000 to 1,000,000.

15. A method according to claim 14, wherein the membrane has a MWCO of about 150,000.

16. A method according to claim 14, wherein a centrifugation or filtration is performed prior to the ultrafiltration.

17. A method according to claim 16, wherein the centrifugation is performed at a rate of about 2500 rpm for about 15 minutes.

18. A method according to claim 16, wherein the filtration material is in the range of 85-100 g/m².

19. A method according to claim 16, wherein a microfiltration is performed after the centrifugation or filtration, but before the ultrafiltration, wherein the microfiltration is performed using a membrane with a pore diameter of about 0.2-1μ.

20. A method according to claim 6, further comprising: (c₁) removing the alcohol portion of the filtrate resulting from ultrafiltration.

21. A method according to claim 20, further comprising: (e) hydrating the residue after alcohol and (e₁) passing the resulting solution through an adsorptive resin.

22. A method according to claim 21, wherein the adsorptive resin is selected from: ethylvinylbenzene-divinylbenzene copolymer, non-ionic styrene-divinylbenzene copolymer; ionic polystyrene; non-ionic polystyrene; nonionic ethylvinylbenzene-divinylbenzene copolymer; and ionic styrene-divinylbenzene copolymer.

23. A method according to claim 22, wherein the adsorptive resin is XAD16 divinyl benzene copolymer resin.

24. A method according to claim 21, further comprising: (e₂) washing the adsorptive resin with water after completion of procedure (e₁).

25. A method according to claim 24, wherein the water washing is performed with water at a temperature of about 45° C.

26. A method according to claim 24, further comprising: (e₃) washing the adsorptive resin with an aqueous alcohol the water wash of (e₂).

27. A method according to claim 26, wherein the alcohol washing is performed with aqueous ethyl alcohol at a temperature of about 30-70° C., the aqueous ethyl alcohol solution being about 70-100% ethyl alcohol by volume.

28. A novel food ingredient, dietary supplement, cosmetic, or pharmaceutical composition, comprising: an isoflavone-phospholipid molecular complex according to claim 1.

29. A composition according to claim 28, wherein about 0.1-3000 mg of the complex are administered daily.

30. A composition according to claim 29, wherein about 1-500 mg of the complex are administered daily.

31. A method of treating a disease, comprising: administering a therapeutically effective amount of an isoflavone-phospholipid molecular complex according to claim 1 to a patient in need thereof, wherein the disease is selected from: premenstrual syndrome, including fluid retention, cyclical mastalgia, and dysmenorrhoea; menopausal syndrome including hot flushes, anxiety, depression, headaches, mood swings, night sweats, and urinary incontinence; Buergers Disease; Reynaud's Syndrome; Reynaud's Phenomenon; angina pectoris; coronary artery spasm; migrane headaches; hypertension; benign prostatic hypertophy; breast cancer; endometrial cancer; large bowel cancer; ovarian cancer; prostatic cancer; testicular cancer; uterine cancer; atherosclerosis; Alzheimers disease; dementia; cognitive dysfunction; inflammatory diseases including inflammatory bowel disease, Crohns disease, ulcerative colitis; alcoholemia; cirrhosis; delirium tremens; osteoporosis; rheumatic diseases including rheumatoid arthritis; acne; baldness including male pattern baldness (alopecia hereditaria); psoriasis and diseases associated with oxidant stress including cancer, myocardial infarction stroke, arthritis, sunlight induced skin damage, and cataracts.

32. A method of treating or reducing the predisposition to symptoms associated with a disease, comprising: administering a therapeutically effective amount of an isoflavone-phospholipid molecular complex according to claim 1 to a patient in need thereof, wherein the disease is selected from: menopause, cancer, Alzheimer's disease, atherosclerosis, hypercholesterolemia, dementia, cognitive dysfunction, osteoporosis, pre-menstrual syndrome, prostate diseases, and alcoholemia.
